(19) United States
(12) Patent Application Publication
(10) Pub. No.: US 2009/0041724 A1
(43) Pub. Date: Feb. 12, 2009

(54) Protein Delivery System

(76) Inventor: Steen Lindkaer Jensen, Lyngby (DK)

Correspondence Address: CROWELL & MORING LLP;INTELLECTUAL PROPERTY GROUP, P.O. BOX 14300, WASHINGTON, DC, 20044-4300, US

(21) Appl. No.: 11/791,917

(22) PCT Filed: Dec. 5, 2005
(86) PCT No.: PCT/GB2005/004655

§ 371 Date: May 12, 2008

(30) Foreign Application Priority Data
Dec. 3, 2004 (GB) 0426641.7

(51) Int. Cl. A61K 35/76; C12N 7/00; C12N 15/00; A61P 11/00; C12N 5/06

(52) U.S. Cl. 424/93.2; 435/235.1; 435/325; 435/236

(57) ABSTRACT

The present invention relates to a virus-like particle (VLP) having a plasma membrane-derived lipid bilayer envelope, said VLP further comprising a viral structural protein, or fragment or derivative thereof, capable of forming an enveloped VLP, a fusiogenic protein and a recombinant target protein; methods for the delivery of recombinant target proteins to cells using said VLP, therapeutic methods using said VLP, compositions and kits comprising said VLP, methods of producing said VLP, and vectors and host cells for producing said VLP are also described.

The present invention relates to the delivery of non-genic substances to cells. In particular the delivery of proteinaceous substances to cells.

At present, gene therapy of genetic or acquired diseases such as cystic fibrosis or cancer generally involves one of two approaches to delivery of the therapeutic sequence. The first approach uses naked nucleic acid or non-viral vectors which are generally liposome-encapsulated or lipid-complexed. The second approach uses viral vectors. Viral vectors can be non-integrative, like adenovirus (Ad) or herpesvirus (HSV), or integrative, like adeno-associated virus (AAV) and retroviruses (e.g. MLV). In the case of Ad and HSV the expression of the therapeutic gene is only transient. In the case of integrative vectors, retroviruses or AAV, there is a long-term (and theoretically cell-life time) expression.

These gene therapy approaches have several drawbacks. The efficiency of transfer of naked nucleic acid or liposome-complexed nucleic acid to cells is extremely poor. Retroviral vectors can integrate in oncogenic regions of the host genome, and this integration can result in leukemia and/or cancer. Non-integrative vectors have a lower risk of oncogenicity however they can only ensure a transient expression of therapeutic genes and hence are only of transient therapeutic benefit. Viral vectors inevitably induce neutralizing antibodies or meet pre-existing antibodies in their hosts and this limits the efficiency of gene transfer and the life-time of transduced cells. All viral vectors, even replication-defective ones, have the theoretical possibility to revert back to a replicative form, and/or to recombine with another virus of the same or related family present at the same time in the same host. However improbable this may be, such an event may result in new virus species with unknown replication capacity, pathogenicity and propagation potential. Finally, all these techniques involve the transfer to recipient cells of genetic material in general and DNA in particular. Transfer of such material is associated with biological risks and thus requires careful consideration of biosafety.

To address these issues safer and more efficient synthetic vectors for nucleic acid transfer are being developed. Viral vectors are being modified to reduce their immunological impact and to control their integration sites in host cells. Stem cell technology combined with ex vivo gene therapy is also being developed.

There are many scenarios, for instance during cell differentiation or tissue development, where only a transient expression of a particular gene is needed. For example, the formation of lung alveoles in humans is a post-natal phenomenon which can be altered by several types of aggressions occurring during this period of time. Such aggressions include oxygen treatment, mechanical hyperventilation, bacterial or viral infections. Broncho-pulmonary dysplasia (BPD) is a pathology of the alveolar development which represents the major complication to respiratory distress in premature newborn infants. Abnormal proliferation of alveolar epithelial cells and abnormal development of pulmonary microvascularisation play an essential role in BPD physiopathology. Keratinocyte Growth Factor (KGF), which stimulates the alveolar epithelial proliferation and controls apoptosis, and Vascular Endothelial Growth Factor (VEGF), which controls the microvascularisation process, are the two major factors involved in normal alveolar development and the expression of these factors have been found to be diminished in BPD. These factors are therefore potential targets for gene therapy. The transient nature of alveolar development results in a short therapeutic window, particularly suitable to the transient transgene expression carried out by a non-integrative vector such as adenovirus.

Other examples of transient gene expression are common during tissue differentiation and proliferation, two phenomena in which cellular genes or growth factor receptors are activated and deactivated at certain times of the cell cycle, resulting in a finely regulated sequence of events.

In many cases where a single genetic defect is found in a single transiently expressed protein, the transient expression of the wild type allele of a mutated gene could be sufficient to correct entire pathologies induced by this genetic defect.

An alternative approach is the direct transfer of the non-defective bioactive gene products, peptides or/and proteins in their native conformation (Ford et al., Gene Ther. 2001,8:1-4; Dalkara et al., Mol. Ther. 2004, 9:964-969). Although the transferred agents will not persist to the same degree as nucleic acid based vectors, they should be able to persist for a sufficient length of time to have efficacy in the transient therapeutic window.

Delivery of therapeutic proteins to cells has certain advantages over delivery of nucleic acid. Since these proteins can be selected to carry correct post-translational modifications if any (e.g. glycosylation, phosphorylation) and can be of the same origin as the host, they will be well tolerated by the hosts and should not induce any immunogenic reaction. This will allow for iterative administrations. As discussed above, a major drawback of virus based gene therapy is the immunogenicity of the vectors. Not only could the vector provoke an immediate adverse reaction to itself, immune protection can develop over time to the extent that repeated administration of the viral vector becomes useless. There is also no possibility of oncogenic integration of a therapeutic protein, which is not the case for all viral vectors. Furthermore, the delivery of a therapeutic protein means the cell machinery of transcription, translation and posttranslational modifications and intracellular trafficking/targeting to a specific cellular compartment (e.g. plasma membrane in case of receptors and cell surface molecules, or nucleus for nuclear factors) is bypassed. This is predicted to minimise stress to the cell.

Although desirable, delivery of proteins to cells in sufficient amounts is difficult to achieve. Liposome mediated techniques have been attempted. Owais, et al. (Eur. J. Biochem, 2000, Vol 267: 3946-3956) have used fusiogenic lipids in liposomes to promote fusion with recipient cells and result in delivery of encapsulated protein. In U.S. Pat. No. 5,631,237 Sendai virus proteins were used to promote the fusion of liposomes to recipient cells.

The present invention takes an alternative approach to the problem of the delivery of non-genic material to cells. In particular, the present invention exploits virus-like particles (VLPs) as delivery vehicles for membrane bound and non-membrane bound proteins.

VLPs are structures resembling a virus particle but devoid of the viral genome. Accordingly they are incapable of replication and devoid of pathogenicity. The particle typically comprises at least one type of structural protein from a virus. In most cases this protein will form a proteinaceous capsid (e.g. VLPs comprising a retrovirus, adenovirus or bacteriophage structural protein). In some cases the capsid will also be enveloped in a lipid bilayer originating from the cell from which the assembled VLP has been released (e.g. VLPs comprising a human immunodeficiency virus structural protein such as Gag).

VLPs are typically formed when a gene encoding a viral structural protein is overexpressed in a host cell in isolation from other viral genes. In the cytosol, the structural proteins assemble into the VLP in a process analogous to the process in which a bona fide virus particle assembles. Of course, without the other viral genes being present a true virus particle cannot be formed. Formation of VLPs results in their release from the host cell. This may be by cell lysis. In the case of VLPs derived from enveloped viruses this process is by budding from the host cell and this results in the VLP being enveloped by a lipid bilayer.

It has now been found that enveloped virus-like particles can be engineered to be fusiogenic and thus capable of delivering both membrane bound and non-membrane-bound proteins to cells.

Thus, in one aspect the present invention provides a virus-like particle (VLP) having a plasma membrane-derived lipid bilayer envelope, said VLP further comprising:

a) a viral structural protein, or fragment or derivative thereof, capable of forming an enveloped VLP;

b) a fusiogenic protein; and

c) a recombinant target protein.

The term “plasma membrane-derived lipid bilayer envelope” refers to a lipid bilayer derived from the plasma membrane of the host cell from which the VLP has been released. This envelope either partially or totally encloses the VLP. The VLP is preferably completely (or substantially completely) enclosed within the envelope. The lipid bilayer will have a macromolecular composition corresponding to the composition of the plasma membrane of the host cell. The bilayer will have similar proportions of the same lipids, proteins and carbohydrates. Such macromolecules would include transmembrane receptors and channels (such as receptor kinases and ion channels), cytoskeletal proteins (such as actin), lipid or protein linked carbohydrates, phospholipids (such as phosphatidylcholine, phosphatidylserine and phosphatidylethanolamine), and cholesterol. The composition will be complex and distinct from the typical composition of artificial bilayer preparations such as liposomes. Liposomes typically comprise a small number of different types of lipids. They are formed spontaneously after sonification of a suspension of these lipids in an aqueous solution. The liposomes will encapsulate part of the aqueous solution and so liposomes can be packed with substrates of interest by including those substrates in the aqueous solution. Liposomes can also be produced with transmembrane proteins embedded in the bilayer however technical limitations mean that the numbers and variety of proteins that can be included in liposomes is severely curtailed. Thus, a liposome bilayer is viewed by the skilled man as distinct in terms of complexity from the plasma membrane derived lipid bilayer of the VLPs of the invention.

Furthermore the spontaneous nature of liposome formation results in a largely disordered and homogenous arrangement of components (i.e. lipids and proteins). Conversely, the components of the lipid bilayer of the VLPs of the invention will be reasonably ordered, as a result of being derived from a plasma membrane which inherently has a degree of order to it.

In a preferred embodiment the plasma membrane-derived lipid bilayer of the VLP of the invention comprises at least four, more preferably at least six most preferably at least eight different lipid types. Preferably on lipid type is phosphatidylserine. Preferably the lipid bilayer of the VLPs of the invention comprises 20% to 60% phosphatidylserine, more preferably 30% to 50% and most preferably about 40%.

In another preferred embodiment the plasma membrane-derived lipid bilayer of the VLP of the invention comprises at least four, more preferably at least six most preferably at least eight different types of protein.

By “virus-like particle” it is meant a structure resembling a virus particle but devoid of the viral genome, incapable of replication and devoid of pathogenicity. The particle typically comprises at least one type of structural protein from a virus. Preferably only one type of structural protein is present. Most preferably no other non-structural component of a virus is present. The VLPs of the present invention include a plasma membrane-derived lipid bilayer envelope.

A “viral structural protein” is a protein that contributes to the overall structure of the capsid protein or the protein core of a virus. The viral structural protein of the present invention can be obtained from any virus which can form enveloped VLPs. These are typically proteins from viruses that are naturally enveloped. Such viruses include, but are not limited to, the Retroviridae (e.g. HIV, Moloney Murine Leukaemia Virus, Feline Leukaemia Virus, Rous Sarcoma Virus), the Coronaviridae, the Herpesviridae, the Hepadnaviridae, and the Orthomyxoviridae (e.g. Influenza Virus). However, naturally non-enveloped viruses may form enveloped VLPs and these are also encompassed by the invention. Naturally non-enveloped viruses include the Picomaviridae, the Reoviridae, the Adenoviridae, the Papillomaviridae and the Parvoviridae.

Preferred structural proteins are the Retroviridae Gag proteins. Particularly preferred as the structural protein is the protein corresponding to the HIV-1 gag gene. This is because the production and assembly of Gag VLPs is highly efficient and these VLPs have low cytotoxicity. The gag gene of the lentivirus HIV-1 codes for the polyprotein Pr55Gag which is a precursor of the structural proteins p17 matrix (MA), p24 capsid (CA), p7 nucleocapsid (NC) and p6. Gag is cleaved into the individual proteins in mature, infectious virions of HIV-1, however, in Gag VLPs Gag remains as a single protein since the required viral protease is absent. The mechanisms underlying and proteins involved in Gag VLP formation are extensively discussed in the prior art (see Carrière et al., 1995 J. Virol. 69:2366-2377; Wilk et al., 2001 J. Virol. 75:759-77130; US2002/0052040; Chazal and Gerlier, 2003 Microbiol. Molec. Biol. Rev. 67:226-237; Hong and Boulanger, 1993 J, Virol. 67:2787-2798; Royer et al., 1992 J. Virol. 66:3230-3235; Spearman et al, 1994 J. Virol. 68:3232-3242 and references cited therein).

Thus, in a preferred embodiment the present invention provides a VLP having a plasma membrane-derived lipid bilayer envelope, said VLP further comprising:

a) HIV1 Gag, or fragment or derivative thereof, capable of forming an enveloped VLP;

b) a fusiogenic protein; and

c) a recombinant target protein.

As in the case of HIV1 Gag, encompassed by the term “structural protein” are pro-structural proteins wherein the structural protein is produced upon post translation cleavage of a pro-protein or structural polyproteins wherein multiple structural proteins are derived from a single polypeptide. These proteins may not need to be cleaved to be able to form a VLP.

Fragments and derivatives of these naturally occurring structural proteins that retain the ability to form VLPs are encompassed by the invention. The skilled man will be aware of how to determine if a particular fragment or derivative retains the ability to form VLPs. For instance see Carrière et al., 1995 J. Virol. 69:2366-2377 and Wilk et al., 2001 J. Virol. 75:759-77130 and references cited therein (for instance Fäcke, et al 1993, J. Virol. 67, 4972-4980) provide direction as to the identification of regions and fragments of Gag that retain the ability to form VLPs. Such technique can be readily applied to other viral structural proteins. These derivatives of naturally occurring sequences will typically have at least 40%, preferably 50 or 60% or more, particularly 70 or 80% or more sequence homology with the native sequence. For the purposes of the present invention, and in accordance with common understanding in the art, “sequence homology” is not used to refer only to sequence identity but also to the use of amino acids that are interchangeable on the basis of similar physical characteristics such as charge and polarity. Substitution of an amino acid within a signal sequence with an amino acid from the same physical group is considered a conservative substitution and would not be expected to alter the activity of the signal peptide. Thus a derivative which just replaced leucine with isoleucine throughout would be considered to have 100% “sequence homology” with the starting sequence. Convenient groups are, glycine and alanine; serine, threonine, asparagine, glutamine and cysteine; lysine arginine and histidine; glutamic acid and aspartic acid; valine, leucine, isoleucine, methionine, phenylalanine, tryptophan and tyrosine. Preferred subgroups within this last group include leucine, valine and isoleucine; phenylalanine, tryptophan and tyrosine; methionine and leucine. Sequence homology may be calculated as for ‘sequence identity’ discussed below but allowing for conservative substitutions as discussed above.

Preferably, the derivatives of naturally occurring virus structural proteins or active fragments thereof exhibit at least 50%, preferably at least 60% or 70%, e.g. at least 80% sequence identity to a naturally occurring structural protein or portion thereof (as determined by, e.g. using the SWISS-PROT protein sequence databank using FASTA pep-cmp with a variable pamfactor, and gap creation penalty set at 12.0 and gap extension penalty set at 4.0, and a window of 2 amino acids.

Naturally occurring structural proteins, or fragments or derivatives thereof, may be provided as a fusion protein with one or more domains of structural proteins belonging to different species, subgroups families or subfamilies of viruses (e.g. Lentivirus and spumavirus; see Carrière et al., supra), or with non-viral protein sequences.

The VLP will typically comprise multiple copies of the viral structural protein (Briggs, J. A., et al., 2004. Nat. Struct. Mol. Biol. 11:672-675). Preferably the VLP will comprise at least 2000 copies of the viral structural protein, more preferably at least 3000 copies and most preferably at least 4000 copies.

The term “fusiogenic protein” means a viral protein that can induce the fusion of the plasma membrane derived envelope of the VLP to the membrane of the recipient cell. It is this mechanism that results in entry of the proteinaceous component of the VLP to the cytosol. The envelope glycoproteins of RNA viruses and retroviruses are well known to bind cell receptors and induce this fusion. Accordingly these proteins are responsible for the infectivity of these viruses. Other examples of fusiogenic proteins include, but are not limited to, influenza haemagglutinin (HA), the respiratory syncytial virus fusion protein (RSVFP), the E proteins of tick borne encephalitis virus (TBEV) and dengue fever virus, the E1 protein of Semliki Forest virus (SFV), the G proteins of rabies virus and vesicular stomatitis virus (VSV) and baculovirus gp64 (Guibinga G H & Friedmann T., 2004, Mol. Ther. 11: 645-651). Functionally equivalent fragments or derivatives of these proteins may also be used. The functionally equivalent fragments or derivatives will retain at least 50%, more preferably at least 75% and most preferably at least 90% of the fusiogenic activity of the wild type protein.

Particularly preferred is the envelope glycoprotein from the Vesicular Stomatitis Virus (VSV-G). VSV-G has high fusiogenic activity and virtually all mammalian cells can bind VSV-G, via the carbohydrate moiety of their plasma membrane glycoproteins. Without wishing to be being bound by theory, the molecular mechanism of VSV-G-cell surface interaction consists of attachment, followed by a step of membrane fusion between the membrane of the cell and the viral envelope. This process has been well documented for the influenza virus haemagglutinin and host cell plasma membranes (Hunter, E. 1997. Viral entry and receptors, in Retroviruses. Cold Spring Harbor Laboratory Press, New York.).

Thus, in another preferred embodiment the present invention provides a VLP having a plasma membrane-derived lipid bilayer envelope, said VLP further comprising:

a) a viral structural protein, or fragment or derivative thereof, capable of forming an enveloped VLP;

b) the envelope glycoprotein from the Vesicular Stomatitis Virus (VSV-G), or fragment or derivative thereof, capable of promoting fusion of the envelope and the recipient cell membrane; and

c) a recombinant target protein

Preferably the fusiogenic protein is heterologous to the viral structural protein. The term heterologous refers to the biological source of the proteins in question and the fact that the each protein source has a different biological source. For instance if the structural protein is an HIV 1 structural protein the fusiogenic protein is not an HIV1 fusiogenic protein.

In a particularly preferred embodiment the present invention provides a VLP having a plasma membrane-derived lipid bilayer envelope, said VLP further comprising:

a) HIV1 Gag, or fragment or derivative thereof, capable of forming an enveloped VLP;

b) the envelope glycoprotein from the Vesicular Stomatitis Virus (VSV-G), or fragment or derivative thereof, capable of promoting fusion of the envelope and the recipient cell membrane; and

c) a recombinant target protein.

The fusiogenic protein may be a fragment or a derivative of one of the aforementioned naturally occurring proteins. These derivatives of naturally occurring sequences will have at least 40%, preferably 50 or 60% or more, particularly 70 or 80% or more sequence homology with the native sequence.

Preferably, the derivatives of naturally occurring virus fusiogenic proteins or active fragments thereof exhibit at least 50%, preferably at least 60% or 70%, e.g. at least 80% sequence identity to a naturally occurring fusiogenic protein or portion thereof (as determined by, e.g. using the SWISS-PROT protein sequence databank using FASTA pep-cmp with a variable pamfactor, and gap creation penalty set at 12.0 and gap extension penalty set at 4.0, and a window of 2 amino acids.

By recombinant it is meant that the target protein is encoded by a recombinant coding sequence and is not naturally present in the plasma membrane-derived lipid bilayer of the host from which the VLPs are released.

The term “target protein” refers to the protein that is to be delivered to the recipient cell. Such proteins may include proteins not found in the recipient cell, proteins from different species or cloned versions of proteins found in the recipient cell. Preferred target proteins of the invention will be proteins with the same status as that found in the recipient cell expressed in such a way that post-translational modification is the same as that found in the recipient cell. Such modification includes glycosylation or lipid modification addition of coenzyme groups or formation of quaternary structure. Most preferred will be wild type proteins corresponding to proteins found in mutated form or absent in the recipient cell.

The recombinant target protein may be a membrane protein or a non-membrane protein. Non-limiting examples of membrane proteins include ion channels such as the Cystic Fibrosis Transmembrane conductance Regulator protein (CFTR), receptor tyrosine kinases such as the PDGF-receptor and the SCF-R receptor (Stem Cell Factor Receptor, or c-kit, or CD117), G-protein linked receptors such as adrenoreceptors. Non-limiting examples of non-membrane proteins include cytosolic proteins such as actin, Ras, ERK1/2 and nuclear proteins such as steroid receptors and histone proteins. Preferred membrane proteins for incorporation into the VLPs of the invention are those with a single transmembrane domain (also known as type 1 membrane proteins) and those with short cytoplasmic tails. A particularly preferred example of a type 1 membrane protein is the SCF-R receptor. Although not a type 1 membrane protein, the CFTR protein is also particularly preferred as an example of a membrane protein that may be incorporated into the VLPs of the invention.

Thus, in a most preferred embodiment the invention provides a VLP having a plasma membrane-derived lipid bilayer envelope, said VLP further comprising:

a) HIV1 Gag, or fragments or derivatives thereof, capable of forming an enveloped virus-like particle;

b) the envelope glycoprotein from the Vesicular Stomatitis Virus (VSV-G), or fragment or derivative thereof capable of promoting fusion of the envelope and the recipient cell membrane; and

c) recombinant CFTR

As mentioned above VSV-G is a broad spectrum fusiogenic protein. Should tissue/cell specificity be required a more specific fusiogenic protein can be selected. Alternatively VSV-G could be modified to impart the required degree of specificity. The skilled man would be able to engineer VSV to impart specificity. By way of example two approaches are recited below.

First, at least one specific cell ligand could be genetically inserted into at least one of the accessible loops of the VSV-G. The cell specific ligand(s) will be determined from the available literature on the target cells or tissues, or from direct experiments of phage biopanning on the desired target cells. This type of technique is well known of those skilled in the art (Gaden et al., J. Virol. 2004, 78:7227-7247).

Second, in the case where the insertion of the cell specific ligand would be detrimental to the membrane fusion function of VSV-G, the cell specific ligand could be genetically inserted into one of the flexible loops of HIV-1 EnvGp160, and co-expressed with VSV-G at the surface of the VLP. A recombinant adenovirus, Ad-EnvGp160-L could be used. EnvGp160 is predicated to be readily incorporated into Gag VLPs (Wyma et al., 2000 J. Virol. 74:9381-9387 and Yu et al., 1992 J. Virol. 66:4966-4971).

In another aspect the invention provides a method of delivering a recombinant target protein to a recipient cell said method comprising:

a) providing a VLP as defined above; and

b) exposing the recipient cell to said VLP.

As mentioned above the recombinant target protein may be a membrane bound protein. Plasma membrane-derived lipid bilayer enveloped virus-like particles are released from producer cells by the process of budding and pinching from the plasma membrane. This is a well documented process (Chazal and Gerlier, supra, Spearman et al., supra; and Sakalian and Hunter, 1998 Adv. Exp. Med. Biol. 440:329-339). Upon budding, VLPs are enveloped by some of the host cell's plasma membrane. The envelope therefore contains host cell derived plasma membrane proteins. If the host cell is engineered to express recombinant membrane proteins in the plasma membrane then the recombinant protein will be incorporated in the plasma membrane-derived lipid bilayer enveloped virus-like particle. The delivery of recombinant target membrane protein to recipient cells would then occur via membrane fusion mediated by the fusiogenic protein. To achieve this the VLP carrying the recombinant membrane protein can simply be incubated with recipient cells.

The recombinant target membrane protein (particularly those with multiple transmembrane domains or long cytoplasmic tails) may be modified in order to optimise the incorporation of the target protein into the VLP. For instance, peptide sequences within HIV-1 gp41 can assist incorporation of membrane proteins into HIV-1 Gag based VLPs when those sequences are inserted into a cytoplasmic domain of membrane proteins (Wyma, D. J. et al., 2000, J. Virol. 74: 9381-9387; Lee S. F. et al., 2000, J. Biol. Chem. 275: 15809-15819). A further example of modifications that can be made to optimise incorporation of membrane proteins into VLPs is to insert a peptide sequence that corresponds to the peptide sequence of the linker of activated T-cell (LAT) protein into a cytoplasmic domain of the membrane protein (Alexander M et al., 2004, J. Virol. 78: 1685-1696).

Thus, in a preferred embodiment the recombinant target protein of the VLP of the invention is a membrane protein that has been modified in a cytoplasmic domain with at least one peptide sequence that assists incorporation of membrane proteins into VLPs. In a further preferred embodiment the recombinant target protein is a membrane protein that has been modified in a cytoplasmic domain with at least one peptide sequence from HIV-1 gp41 or LAT that assists incorporation of membrane proteins into VLPS.

The fusion of the VLP membrane (‘donor membrane’) with the recipient cell membrane (‘acceptor membrane’) is induced by the fusiogenic protein and results in the transfer of the recombinant membrane protein to the membrane of the recipient cell. The direct membrane-to-membrane transfer of the recombinant membrane protein to the recipient cell membrane should preserve the local conditions (e.g. plasma membrane biochemical microenvironment, putative partner proteins, etc) necessary for its optimal biological activity and functionality.

If the membrane protein is not normally found in the plasma membrane (e.g. the protein is found in organelle membranes) it is within the capabilities of the skilled man to engineer such proteins to promote their targeting to the plasma membrane instead of their naturally location in the cell.

A protein transfer method such as described above has advantages over gene transfer. Membrane proteins are often complex proteins. The CFTR protein is indicative of such proteins. The CFTR protein is a transmembrane glycoprotein serving as a Cl-channel and is implicated in the disease Cystic Fibrosis (CF). CFTR has multiple transmembrane domains, two intracytoplasmic domains (endodomains) carrying regulated regions, which correspond to the N- and C- termini, and extracellular or ectodomains. Both ectodomain and endodomain are posttranslationally modified by glycosylation and phosphorylation, respectively. The biological activity of the CFTR requires different types of posttranslational modifications and plasma membrane addressing, both features which in turn require a rigorous intracellular folding and a correct three-dimensional structure, and a well-defined intracellular pathway. Furthermore, the length of the coding sequence (over 1,400 amino acid residues) makes it impossible to clone it along with all its regulatory upstream and downstream elements. Considering the complexity of the CFTR protein molecule, it is predictable that the regulated expression of human cftr gene using a viral or non-viral vector will meet with some difficulty. By expressing this protein in a suitable host cell engineered to produce VLPs of the invention, the protein is incorporated in a VLP as a properly expressed and modified CFTR in its correct membrane context. The protein is accordingly administered in the best possible conformation.

To enable delivery of non-membrane bound proteins these proteins are incorporated in the VLP by expressing the target protein as a fusion protein with a protein capable of binding to the viral structural protein. For instance if the viral structural protein is HIV-1 Gag then the N- or C-terminus of the Vpr protein of HIV-1 can be used. Vpr has been shown to interact with the C-terminal p6 domain of Gag, and stoichiometric ratios of Gag and Vpr-X (fusion protein of Vpr and a putative target protein) have been found to be encapsidated (Zhu, et al., 2004 Retrovirology 1:26-31.). Vpr-X will be transported within the core of the Gag particles as a p6-bound internal protein.

Upon fusion between the envelope of the VLP and the recipient cell membrane, the internal core of the VLP will dissociate in the cytosol. This in turn labilizes the protein-protein interactions occurring between the structural protein and the fusion protein (e.g. p6 domain of Gag and the Vpr moiety of the Vpr-X fusion protein) releasing the fusion protein. The natural properties of the protein to be fused to the target protein can be exploited. For instance Vpr contains a nuclear localization signal and is naturally transported to the nucleus. Accordingly, Vpr-tagged proteins will be transported to the nucleus. The skilled man will be aware of potential candidates for these fusion proteins and their properties. By way of example, Vif, an auxiliary protein of the HIV-1 virion, also interacts with Gag and can be efficiently co-encapsidated with Gag (Bardy et al., 2001 J. Gen. Virol. 82:2719-2733.; Bouyac et al., 1997 J. Virol. 71:9358-9365; and Huvent et al., 1998 J. Gen. Virol. 79:1069-1081). Vif does not have a nuclear localisation signal. The recombinant protein to be delivered can therefore be fused to Vif, to result in delivery to cell compartment(s) different from the nuclear compartment mainly targeted by Vpr.

Delivery may be to a cell in vivo, in vitro or ex vivo. The cell may be in isolation, in culture with other cells or in situ in a tissue.

In another aspect the invention provides a method of protein therapy said method comprising:

a) providing a VLP as defined above; and

b) exposing a cell to said VLP in an amount effective to elicit a therapeutic effect associated with the therapeutic protein.

Typically, protein therapy involves the delivery of proteins to cells to achieve a therapeutic effect. Typically the cell will be deficient in the therapeutic protein. By deficient it is meant that the cell does not have sufficient quantities of correctly functioning protein. This may mean that the cell does not express the protein at all but it may also mean that the cell expresses a mutated version of the protein. Through delivery of therapeutic amounts of the protein to a cell deficient in the protein the disease state induced by the deficiency may be reversed. Candidates for such an approach include the CFTR protein.

Thus in a preferred embodiment the invention provides a method of protein therapy for the treatment of cystic fibrosis.

Alternatively the therapeutic protein may be a proteinaceous pharmaceutical and administration is to a cell which is not deficient in the therapeutic protein but a cell that would still benefit from exposure to the therapeutic protein.

The mode of administration of the VLP in a protein therapy method will vary depending on the disease being treated since different diseases will require administration of the VLP at different sites in the body. For instance treatment of cystic fibrosis is likely to involve administration to the airway epithelium of the respiratory tract. This will be advantageously prescribed early in life of CF-patients, in infants and young children, in order to prevent anomalies in the development of lung and respiratory tract, as well as to avoid occurrence of complications inherent to opportunistic infections constantly observed in this disease.

Typically the VLP will be administered in a pharmaceutically acceptable composition.

The present invention therefore also provides a pharmaceutical composition comprising a VLP as defined above together with at least one pharmaceutically acceptable carrier, diluent or excipient.

The VLP in such compositions may comprise from 0.05% to 99% by weight of the formulation, more preferably 0.1% to 10%.

By “pharmaceutically acceptable” is meant that the ingredients must be compatible with other ingredients of the composition as well as physiologically acceptable to the recipient. The pharmaceutical compositions may be formulated according to any of the conventional methods known in the art and widely described in the literature. Thus, the active ingredient may be incorporated, optionally together with other active substances, with one or more conventional carriers, diluents and/or excipients, to produce conventional galenic preparations such as tablets, pills, powders, lozenges, sachets, cachets, elixirs, suspensions, emulsions, solutions, syrups, aerosols (as a solid or in a liquid medium), ointments, soft and hard gelatin capsules, suppositories, sterile injectable solutions sterile packaged powders, and the like. Preferably the composition is adapted for administration by injection or aerosol.

Examples of suitable carriers, excipients, and diluents are lactose, dextrose, sucrose, sorbitol, mannitol, starches, gum acacia, calcium phosphate, aglinates, tragacanth, gelatin, calcium silicate, microcrystalline cellulose, polyvinylpyrrolidone, cellulose, water syrup, water, water/ethanol, water/ glycol, water/polyethylene, glycol, propylene glycol, methyl cellulose, methylhydroxybenzoates, propyl hydroxybenzoates, talc, magnesium stearate, mineral oil or fatty substances such as hard fat or suitable mixtures thereof. The compositions may additionally include lubricating agents, wetting agents, emulsifying agents, suspending agents, preserving agents, sweetening agents, flavouring agents, and the like. The compositions of the invention may be formulated so as to provide quick, sustained or delayed release of the active ingredient after administration to the patient by employing procedures well known in the art.

Alternatively the VLP (or composition) may be administered to cells ex vivo prior to implantation or re-implantation. One application, for instance, will be the transfer of growth factor receptors to stem cells to confer upon them the capacity of multiplication and expansion in vitro before re-administration to the patients from whom they were obtained.

Stem cell growth ex vivo is a difficult process, as the cultures usually stabilize at a plateau level of about 3×10⁹ cells, at which they stop dividing. By transferring a growth factor receptor to plateauing stem cells using the protein transfer method of the invention these cells could be induced to proliferate further.

The SCF-R receptor (Stem Cell Factor Receptor, or c-kit, or CD117) is a putative candidate. Treatment of stem cells previously exposed to VLPs of the invention carrying SCF-R with the specific ligand (SCF) would restimulate the growth of those stem cells. This process could be repeated several times to achieve the level of 10¹¹ stem cells which is necessary for in vivo therapy of liver cancer. Membrane receptors other than SCFR might also function to stimulate the proliferation of stem cells, e.g. EGFR. Alternatively, Hox4 or telomerase might be delivered intracellularly via Vpr or Vif tag, to achieve the same goal via transcriptional activation of stem cell growth.

Another application would be the transfer of specific immunogenic proteins (e.g. tumor antigens) to dendritic cells (DC) ex vivo, using the intracellular protein delivery method of the invention, so that these antigens will be processed into immunogenic peptides and expressed at the cell surface by MHC-class II molecules. MHC-class II presentation of these immunogenic peptides will induce or re-enforce the immune response to tumor cells when treated-DC are re-administrated in vivo.

Yet another ex vivo application of consists of transferring tissue-specific cell surface molecules to stem cells isolated from a patient to ensure retargeting of the stem cells to specific organs upon systemic administration.

In another aspect the invention provides a VLP as defined above for use in therapy. The compositions described above may also be used in therapy. Preferably the therapy involves the treatment of diseases characterised by a protein deficiency, most preferably the treatment of cystic fibrosis.

In another aspect the invention provides the use of a VLP as defined above in the manufacture of a medicament for the treatment of cystic fibrosis.

The VLP of the invention may also be used as a vaccine or in immunotherapy. In this case, the recombinant protein incorporated in the VLP envelope will be selected for its potential to function as an immunogen. The advantage of using VLPs of the invention in a vaccine or immunotherapy is that the recombinant membrane protein will be in its natural context of membrane phospholipids and neighboring proteins, it will have the correct posttranslational modifications (e.g. their glycosylation status) and it will be in its native conformation. These are quasi-ideal parameters for presenting immunogenic epitopes and generating highly reactive and specific antibodies when administered into the desired host.

A typical immunization protocol using the VLPs of the invention would involve an initial administration of soluble protein of interest followed by a boost using a VLP incorporating the protein and then a second boost using synthetic peptides representing the immunogenic epitopes. Variations on this protocol in which steps are inverted, or repeated, is also be envisaged. This type of strategy can be applied to human patients (immunotherapy, vaccination), as well as to laboratory animals, destined to produce highly reactive antibodies for laboratory use in vitro, or for manufacturing diagnostic kits used in human or veterinary medicine.

In addition to the pharmaceutically acceptable carriers, diluents and excipients previously described, the vaccine compositions will further comprise adjuvants. Non-limiting examples include immunostimulatory nucleic acids, peptidoglycans, lipopolysaccharides, lipoteichonic acids, imidazoquinoline compounds, flagellines, lipoproteins, immunstimulatory organic molecules, unmethylated CpG-containing oligonucleotides or mixtures thereof.

VLPs of the invention may also be used to deplete undesirable soluble factors from a solution. VLPs can be engineered to express receptors for the soluble factor to be depleted. The VLP would then sequester the soluble factor from the solution through formation of complexes between the receptor and the soluble factor. A potential application is anti-angiogenesis cancer biotherapy. The vascularisation of tissues in development in general and of tumors in particular is positively controlled by several soluble factors, e.g. VEGF (Vascular Endothelial Growth Factor) or other angiogenesis factors. Any blockage of the interaction between VEGF and its specific receptors (VEGF-R) at the surface of endothelial cells will result in a lower level of tumor vascularisation, and regression of the tumor by the resulting anoxia. VLPs carrying the membrane-inserted and surface-exposed VEGF-R (or any receptor for other angiogenesis factors) will compete with the same receptors expressed at the surface of the endothelial cells. Intratumoral injections of such VLPs will deplete the extracellular medium from circulating VEGF (or other angiogenesis factors), and block the neovascularisation, and hence the expansion of the tumor.

The skilled man will readily understand how these therapeutic techniques can be applied to in vitro scenarios and thus the invention is of great utility as a in vitro research tool for the delivery of proteins of interest to experimental systems.

As discussed above, the VLPs of the invention form when overexpressed viral structural proteins assemble and bud from a host cell.

Thus, in another aspect the invention provides a method for the production of a VLP as defined above, said method comprising the coexpression of a viral structural protein, or fragment or derivative thereof, capable of forming an enveloped VLP, together with a fusiogenic protein, together with a recombinant target protein in an in vitro cultured cell and isolating the VLP from the culture media.

The host cell can be any cell, preferably eukaryotic, and more preferably mammalian. Most preferably the source of the cell will be the same or compatible with the cell to which the VLP are designed to fuse with. Preferably the cell is in a stable cell culture.

In some cases, nucleic acids encoding (i) a viral structural protein, or a fragment or derivative thereof, capable of forming an enveloped VLP, (ii) a fusiogenic protein, and/or (iii) a recombinant target protein can be stably integrated individually, pairwise or together into the genome of a cell leading to a stable cell line capable of continuous growth in vitro. Such a cell line will preferably express constitutively the VLP or constituents thereof.

In another aspect the invention provides an in vitro host cell line comprising

a) a nucleic acid encoding a viral structural protein, or fragment or derivative thereof, capable of forming an enveloped VLP;

b) a nucleic acid encoding a fusiogenic protein; and

c) a nucleic acid encoding a recombinant target protein.

Another aspect of the invention is a nucleic acid vector comprising

a) a sequence encoding a viral structural protein, or fragment or derivative thereof, capable of forming an enveloped VLP;

b) a sequence encoding a fusiogenic protein; and

c) a cloning site in which the coding sequence of a target protein can be inserted.

Such vectors incorporating sequences which encode target proteins are a further aspect of the invention; the expression products of said vectors being capable of forming a VLP as described above. Such vectors may be derived from any known viral vector (e.g. adenovirus, AAV, HSV, vaccinia virus or baculovirus vectors) or non-viral vectors (e.g. plasmids or yeast artificial chromosomes).

In a preferred embodiment the vector further comprises at least one from the following list in positions that allow for the true functioning of the sequence; an origin of replication, a selectable marker, a transcriptional start site, a transcriptional enhancer, a transcriptional inducer, a transcriptional control element, a 3′ untranslated control sequence, a 5′ untranslated control sequence and sequences to allow for detection and/or purification of the target protein product. The choice of the particular additional sequences will be dependent on the host cell type.

In a further aspect the invention provides a kit comprising a vector as defined above and a (engineered) host cell line. Alternatively the kit can comprise

(a) a stable host cell line engineered to comprise a nucleic acid encoding a viral structural protein, or fragment or derivative thereof, capable of forming an enveloped VLP and a nucleic acid encoding a fusiogenic protein; and

(b) a vector suitable for eventual transfection of the host cell line with a sequence encoding the target protein. Preferred embodiments recited for specific aspects apply mutatis mutandis to all aspects.

In another aspect the invention provides a VLP having a plasma membrane-derived lipid bilayer, said VLP further comprising at least one viral structural protein, or fragment or derivative thereof, capable of forming an enveloped virus-like particle and a recombinant membrane bound target protein, wherein the target protein is located in the envelope of the virus-like particle.

Any and all preferred viral structural proteins and membrane bound target proteins discussed above apply to this aspect of the invention.

Previous discussion of compositions, host cells, methods of production, medical uses, kits and vectors apply mutatis mutandis to this aspect of the invention.

The VLPs of this aspect of the invention are particularly applicable to vaccination or immunotherapy protocols, vaccine compositions and protocols designed to deplete a solution of an undesirable soluble factor. Accordingly the previous discussion of these aspects of the invention in relation to fusiogenic VLPs is equally applicable to the VLPs of this aspect of the invention.

The invention will be further described with reference to the following non-limiting Example in which:

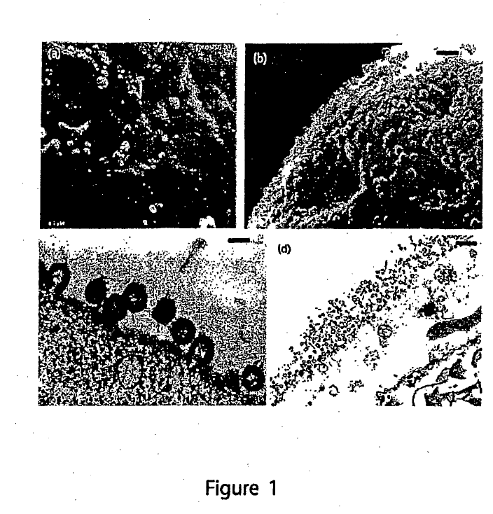

FIG. 1. Electron microscopic (EM) analysis of control Sf9 cells (a), and baculovirus-infected Sf9 cells (b-d), demonstrating the high efficiency of budding and high production of Gag vehicles by insect cells infected in vitro with the recombinant baculovirus AcMNPV-Gag. (a, b), Scanning electron microscopic images of the cell surface, showing relatively smooth external side of the plasma membrane of noninfected cells (a), contrasting with the ‘bubbles’ or (‘buds’) visible in number at the surface of Gag-expressing cells (b). These buds correspond to Gag VLP, as observed in ultrathin sections (c, d). (c, d), Transmission EM analysis of ultrathin sections of AcMNPV-Gag-infected cells, shown at two different magnifications. The electron-dense crown of material underlying the membrane double leaflet is constituted of Pr55Gag polyprotein molecules constituting the inner core of the Gag particle. Bar represents 500 nm in (a) and (b), 100 nm in (c), and 1 mm in (d).

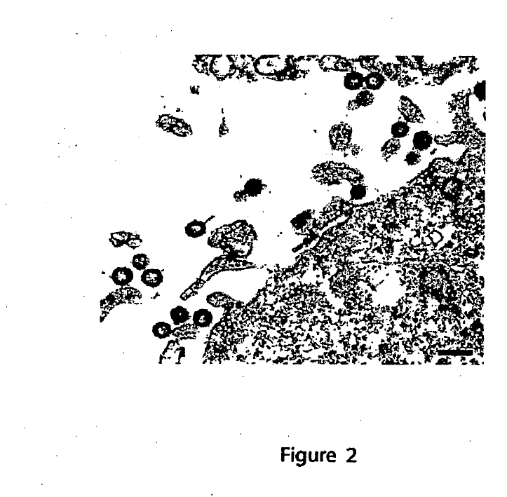

FIG. 2. Transmission EM analysis of sections of mammalian cells infected with a recombinant, Gag-expressing human adenovirus, Ad5-Gag. Note the Gag particles released in the extracellular medium. Bar represents 200 nm.

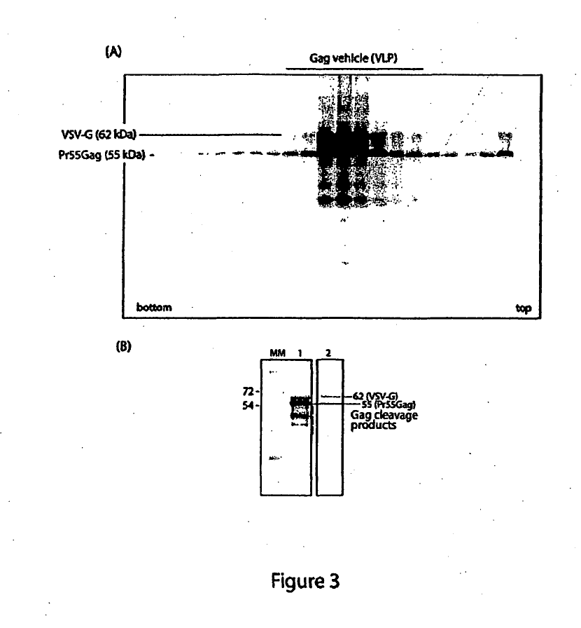

FIG. 3. Pseudotyping of Gag vehicles with viral glycoprotein VSV-G. SDS-PAGE and immunoblot analysis of Gag particles (VLP). Purified Gag particles were denatured by SDS-, electrophoresed in denaturing polyacrylamide gel (PAGE), proteins transferred to nitrocellulose membranes, and detected with monoclonal antibodies (mAb) anti-Gag or anti-VSV-G. (A), Ultracentrifugation analysis in sucrose-D₂O gradient of VSV-G-pseudotyped Gag vehicles. Blot was simultaneously reacted with anti-Gag and anti-VSV-G mAbs. Note the co-sedimentation of the Pr55Gag and VSV-G 62-kDa signals.

(B), Immunoblot analysis of the peak of Gag particles obtained in (A) using anti-Gag (lane 1), or anti-VSV-G (lane 2) mAb; MM, prestained molecular weight markers. The dotted line on the right side of lane 1 indicates Gag spontaneous cleavage products.

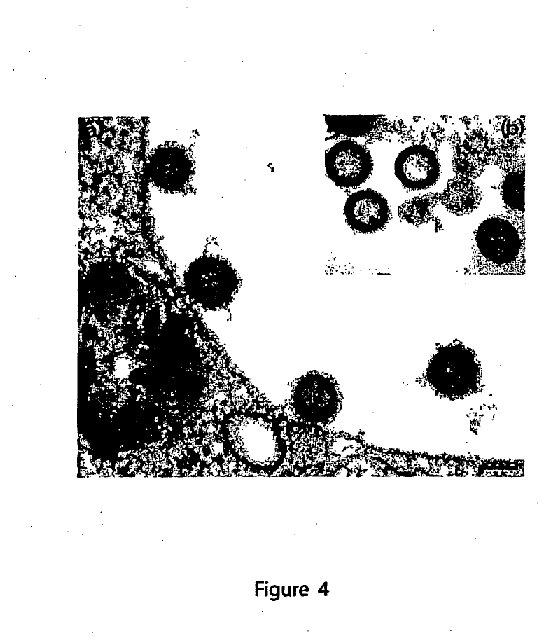

FIG. 4. EM analysis of pseudotyping of Gag vehicles with VSV-G. (a), Gag particles budding from Sf9 cell co-expressing Pr55Gag and VSV-G. (b), Control, Gag particles budding from Sf9 cell single expressing Pr55Gag. Note the difference in the morphology of membrane-enveloped particles, between ‘smooth’ Gag particles shown in (b), and ‘fluffy’ Gag particles shown in (a): the ‘fluffy’ aspect most likely corresponds to membrane-inserted VSV-G molecules, as evidenced in western blot of FIG. 3.

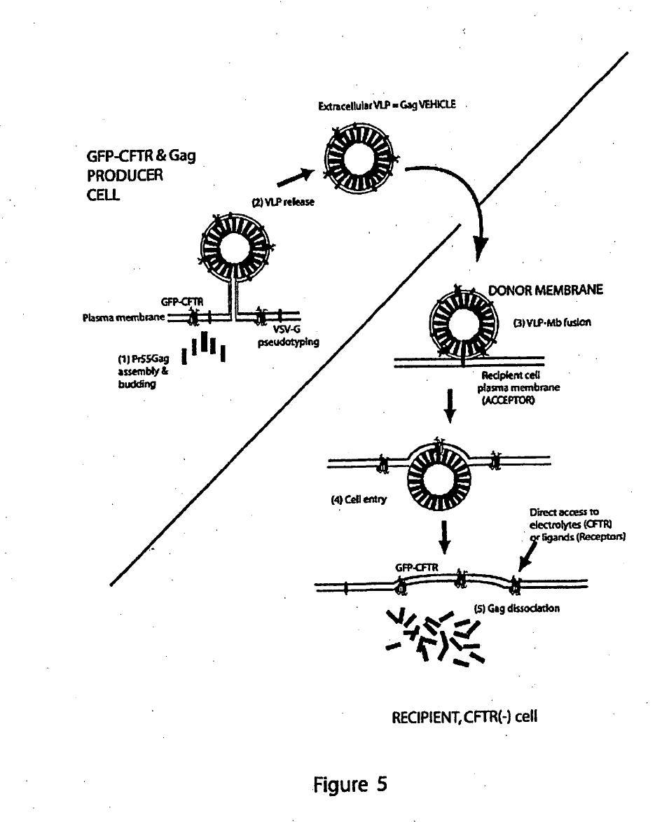

FIG. 5. Principle of the non-genic, membrane-to-membrane protein delivery. Represented schematically in the top left corner of the panel, is the producer cell, expressing Pr55Gag, VSV-G glycoprotein (as the membrane-fusiogenic, pseudotyping agent), and the target protein to be transferred (i.e. the Cl-channel/CFTR molecule, tagged with GFP (GFP-CFTR)). After assembly and budding, and extracellular release, the VSV-G-pseudotyped Gag particles carrying membrane-inserted GFP-CFTR molecules will be incubated with target cells in vitro, as depicted in the lower right corner of the panel. VSV-G will induce the fusion between the pseudo-viral membrane ‘donor membrane’ with the plasma membrane of the recipient cell (‘acceptor membrane’). Upon membrane fusion, GFP-CFTR proteins and their surrounding microdomain will be transferred to the target cell plasma membrane. GFP-CFTR, as any other cell surface molecule or receptor, will then be directly available and accessible to their soluble ligands with no further cellular process.

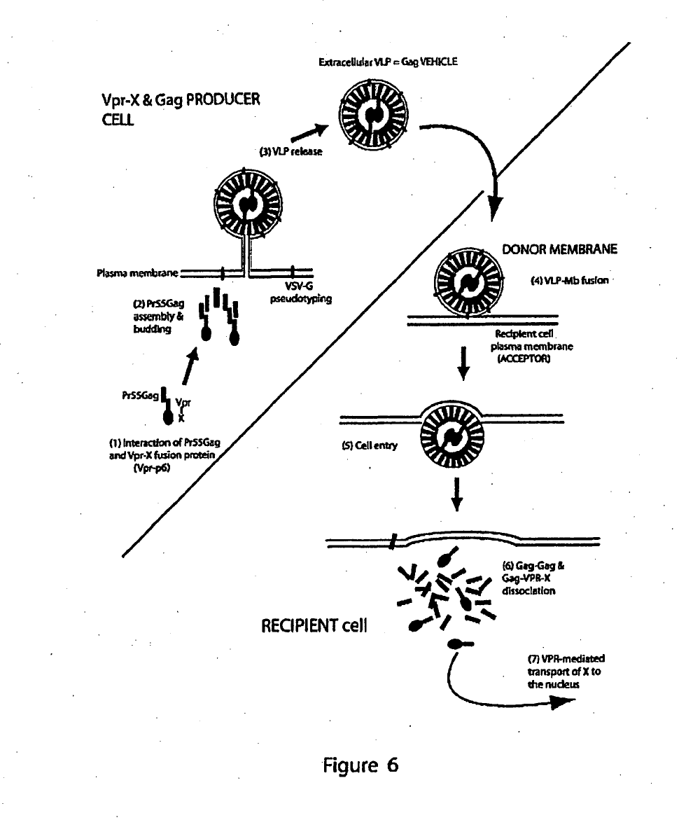

FIG. 6. Principle of the non-genic, Gag-Vpr-mediated intracellular protein delivery. Represented schematically, in the top left corner of the panel, is the producer cell expressing Pr55Gag, the VSV-G glycoprotein (as the membrane-fusiogenic, pseudotyping agent), and the Vpr-fused target protein (Vpr-X) to be transferred. After interaction of Gag-Gag and Gag-Vpr-X (via the p6 domain of Pr55Gag) and assembly of particles, the VSV-G-pseudotyped Gag particles released by extracellular budding will be incubated with target cells in vitro, as depicted in the lower right corner of the panel. VSV-G will induce the fusion between the pseudo-viral membrane ‘donor membrane’ with the plasma membrane of the target cell (‘acceptor membrane’). After membrane fusion, the Gag particle will dissociate within the cytosol of the target cell, and Vpr-X fusion protein will be transferred to the nucleus, via the Vpr-NLS-mediated pathway.

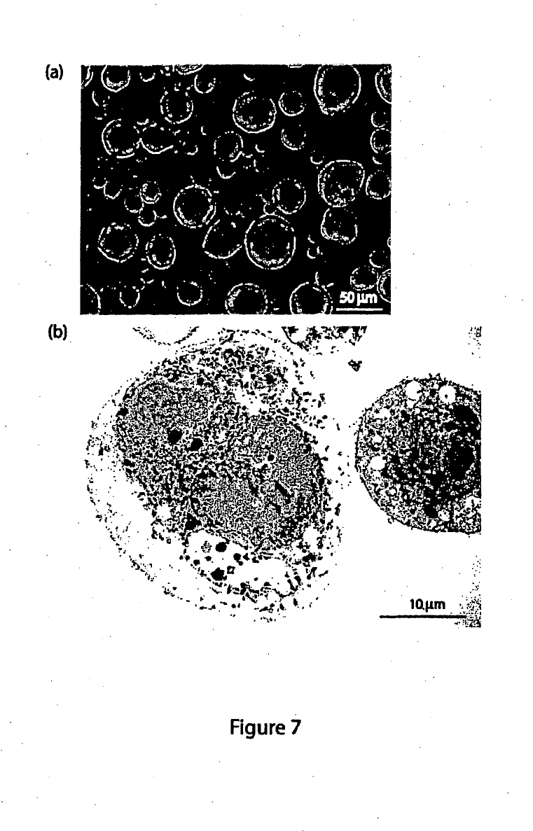

FIG. 7. Insect cell syncytium induced by co-expression of retroviral Gag polyprotein and non-retroviral fusiogenic VSV-G glycoprotein, both localised at the plasma membrane and co-encapsidated in membrane-enveloped virus-like particles. A) Phase contrast microscopy, note the aspects of “rosettes” preceding the cell-cell fusion. B) Electron microscopy, note the giant multinuclear cell on the left, the normal single-nuclear cell on the right.

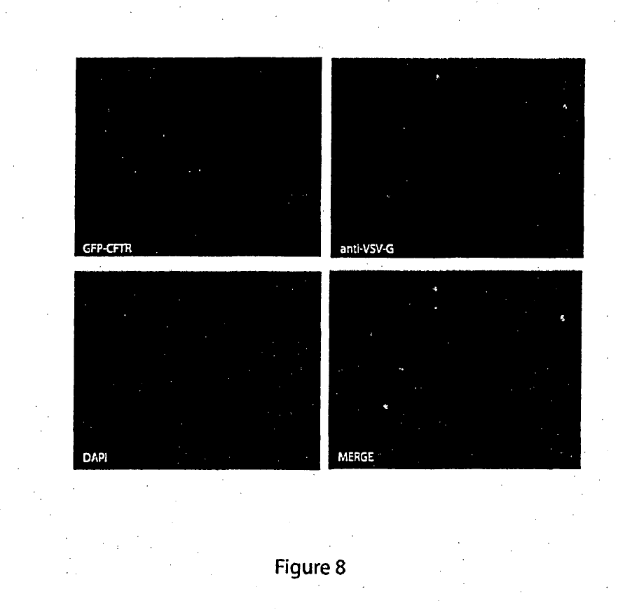

FIG. 8. Transfer of GFP-CFTR to human A549 cells, mediated by Gag VLPs comprising VSV-G glycoprotein. GFP signal is seen at the plasma membrane and co-localises with VSV-G as visualised by RITC-labelled anti-VSV-G antibody. DAPI counterstaining of nuclei is shown on the bottom left panel.

### EXAMPLES

### Example 1

Production of CFTR-GFP VLPs

A chimeric gene, which codes for the green fluorescence protein (GFP) fused to CFTR with the GFP domain at the N-terminus of the CFTR protein was incorporated into VLPs. The GFP moiety enables rapid identification the tagged CFTR and enables the location of the tagged protein to be rapid established. The fused gene gfp-cftr was been cloned in two different expression systems, (i) the baculovirus AcMNPV, to generate the recombinant baculovirus AcMNPV-GFP-CFTR, and (ii) in the adenovirus type 5 (Ad5) to generate the recombinant virus Ad5-GFP-CFTR. The protein was observed to be localised to the cell plasma membrane. A similar GFP-tagged construct, carried by an expression plasmid, has been reported to have the same functionality as the nontagged protein CFTR (Haggie et al., 2002, J. Biol. Chem. 277:16419-16425 and Loffing-Cueni et al., 2001, Am. J. Physiol. Cell Physiol. 281:C 1889-1897.20). The GFP-CFTR clone was shown to be active and functional (Robert et al., 2004, J. Biol. Chem. 279:21160-21168.), as the Ad5-GFP-CFTR clone restored the activity of the Cl-channel in CFTR-deficient cells. As a negative control the biologically inactive mutant of CFTR, CFTRΔF508 was used.

Sf9 cells were co-infected with three recombinant baculoviruses, AcMNPV-Gag, AcMNPV-VSV-G and AcMNPV-GFP-CFTR. Upon budding, Gag virus-like particles carrying VSV-G and GFP-CFTR can be isolated from the culture supernatants using a method of ultracentrifugation in sucrose-D₂0 gradients (Huvent et al., 1998 J. Gen. Virol. 79:1069-1081.) which is well known of those skilled in the art. Briefly, culture media was collected between 60-72 hrs post-infection. The harvested culture media was centrifuged at 2,500 rpm for 10 minutes at 4° C. The supernatant, which contains the VLPs, was collected and the cell pellet was discarded.

The supernatant (approx. 11 ml) was then layered over a sucrose cushion of 1 ml of 20% sucrose in PBS in ultracentrifuge tubes designed to fit into a SW41 Beckman rotor. The tubes were then centrifuged at 30,000 rpm for 1 hr at 4° C. This centrifugation step concentrated the VLPs from a large volume of culture media (approx. 60 ml). The supernatant was discarded and the pellet was resuspended in PBS (200 μl/pellet). Resuspended pellets were left overnight at 4° C. Pellet resuspensions were then pooled for the second centrifugation through a 30-50% sucrose-D₂0 gradient.

The sucrose gradient (30-50% w/v) was made with 50% sucrose solution in deuterium water (D₂0) buffered to pH 7.2 with NaOH, and 30% sucrose solution in 10 mM Tris-HCl, pH 7.2, 150 mM NaCl, 5.7 mM Na₂EDTA. The gradient was made using a gradient mixer in ultracentrifuge tubes (10 ml gradient) designed to fit into the Beckman SW41 rotor. The VLPs (approx. 1.5 ml) were layered onto the sucrose gradient and the tubes were centrifuged at 28,000 rpm at 4° C. for 18 hrs. At the end of the centrifuge run, a thick milky white band and a faint band was observed. The thick band corresponded to the Gag particles while the faint band consisted of mainly baculoviruses. The VLPs were recovered by piercing the tube with a syringe and needle just underneath the thick band and extracting approx. 500 μl of the thick bands.

All of the extracted VLP bands were pooled and were diluted 5 times in PBS. The diluted solution was then centrifuged at 30,000 rpm at 4° C. for 2 hrs to pellet down the VLPs. The supernatant was discarded and the VLPs were resuspended in PBS (200 μl/ml). The resuspended pellet was left at 4° C. overnight. The VLP preparation was then quantitated (by mg protein).

### Example 2

Transmission Electron Microscopy

Cells were infected at a multiplicity of infection (MOI) of 25 with the viruses indicated in the Figure legends. Cells were harvested for electron microscopy (EM) processing at 48 h post infection and pelleted by centrifugation. Cell pellets were then fixed with 2.5% glutaraldehyde in 0.1M phosphate buffer, pH 7.5, post-fixed with osmium tetroxide (2% in H₂O) and treated with tannic acid (0.5% in H₂O). After dehydration, the specimens were embedded in Epon (Epon-812, Fulham, Latham, N.Y.). Sections were taken and stained with 2.6% alkaline lead citrate-0.5% uranyl acetate in 50% ethanol, and examined under a Jeol 1200-EX electron microscope.

### Example 3

Scanning Electron Microscopy

Cells were grown on glass coverslips and were infected at a multiplicity of infection (MOI) of 25 with the viruses indicated in the Figure legends. Fixation and postfixation were as described for transmission EM. Cells were then critical-point dried with liquid CO₂, coated with gold and examined with a Zeiss DSM 950 electron microscope.

### Example 4

Gag VLP Analysis-Ultracentrifugation Analysis

Gag VLPs released from the plasma membrane of Sf9 cells were recuperated and analysed by ultracentrifugation through sucrose-D₂O gradients (Huvent, I. et al., 1998, J. Gen. Virol. 79: 1069-1081). Linear gradients (10 ml total volume, 30-50% w/v) were centrifuged for 18 h at 28,000 rpm in a Beckman SW41 rotor. The 50% sucrose solution was made in D₂O buffered to pH 7.2 with NaOH, and the 30% sucrose solution was made in 10 mM Tris-HCl, pH 7.2, 150 mM NaCl, 5.7 mM Na₂EDTA. Aliquots of 0.5 ml were collected from the top of the tube and the proteins analysed by SDS-PAGE in 10% acrylamide gels using a discontinuous buffer system (Laemmli, 1970, Nature, 227: 680-685).

Gag VLP Analysis-Immunoblot Analysis

Proteins were electrophoresed as described above and electrically transferred to nitrocellulose membranes (Hybond-ECL, Amersham). After blocking with 5% skimmed milk in Tris-buffered saline (20 mM Tris-HCl, pH 7.5, 150 mM NaCl) containing 0.05% Tween 20 (TBS-T) for 1 h at room temperature, the Gag and VSVg proteins were detected by reactions with anti-Gag (laboratory made rabbit polyclonal antibody) or anti-VSVg (monoclonal anti-VSVg, Sigma) antibodies, followed by alkaline phosphatase-labelled anti-IgG conjugate. Colour development of the blots was performed with 5-bromo-4-chloro-3-indolyl phosphate toluidinium and nitroblue tetrazolium (Euromedex).

### Example 5

VLPs-Mediated Protein Transfer and Analysis by Immunofluorescence Microscopy

Gag VLPs were purified from sucrose-D₂O gradients as described above. Purified Gag VLPs were then diluted in DMEM culture media (Invitrogen) and added to A549 cells grown on glass coverslips. The cells were incubated with the VLPs for 1 h at 37° C., and rinsed three times with phosphate buffered saline (PBS). The cells were then fixed with 2% paraformaldehyde in PBS for 10 mins at room temperature. The cells were then blocked with 1% bovine serum albumin in PBS for 30 mins at room temperature and incubated for 1 h with anti-VSVg (1:1000 dilution). The cells were the incubated with a rhodamine-labelled secondary anti-mouse antibody (Sigma) for 1 h at room temperature. The cells were then incubated with DAPI (4′,6-diamidino-2-phenylindole; Sigma) before being mounted on slides for observation using an Axiovert 135 inverted microscope (Zeiss) equipped with an AxioCam digital camera.

1. A virus-like particle (VLP) having a plasma membrane-derived lipid bilayer envelope, said VLP further comprising:
a) a viral structural protein, or fragment or derivative thereof, capable of forming an enveloped VLP;
b) a fusiogenic protein; and
c) a recombinant target protein.

2. The VLP as claimed in claim 1 wherein the plasma membrane-derived lipid bilayer envelope comprises at least four different lipid types.

3. The VLP as claimed in claim 1 wherein the plasma membrane-derived lipid bilayer envelope comprises phosphatidylserine.

4. The VLP as claimed in claim 1 wherein the plasma membrane-derived lipid bilayer envelope comprises at least four, more preferably at least six most preferably at least eight different types of protein.

5. The VLP as claimed in claim 1 wherein the viral structural protein is a structural protein from a virus from a virus family selected from the group consisting of Retroviridae, Coronaviridae, Herpesviridae, Hepadnaviridae, and Orthomyxoviridae.

6. The VLP as claimed in claim 5 wherein the viral structural protein is a structural protein from HIV-1.

7. The VLP as claimed in claim 6 wherein the viral structural protein is HIV-1 Pr55Gag.

8. The VLP as claimed in claim 1 wherein the VLP comprises at least 2000 copies of the viral structural protein.

9. The VLP as claimed in claim 1 wherein the fusiogenic protein is selected from the group consisting of haemagglutinin, the respiratory syncytial virus fusion protein, the E proteins of tick borne encephalitis virus and dengue fever virus, the E1 protein of Semliki Forest virus, the G proteins of rabies virus and vesicular stomatitis virus and baculovirus gp64 or functionally equivalent fragments or derivatives thereof.

10. The VLP as claimed in claim 9 wherein the fusiogenic protein is the G protein of vesicular stomatitis virus.

11. The VLP as claimed in claim 1 wherein the fusiogenic protein is heterologous to the viral structural protein.

12. The VLP as claimed in claim 1 wherein the recombinant target protein is a membrane protein.

13. The VLP as claimed in claim 12 wherein the membrane protein is a membrane protein with a single transmembrane domain.

14. The VLP as claimed in claim 1 wherein the recombinant target protein is selected from the group consisting of CFTR, SCF-R, EGFR and Hox4.

15. A method of delivering a recombinant target protein to a recipient cell said method comprising:
a) providing a VLP as defined in claim 1; and
b) exposing the recipient cell to said VLP.

16. A method of protein therapy said method comprising:
a) providing a VLP as defined in claim 1; and
b) exposing a cell to said VLP in amount effective to elicit a therapeutic effect associated with the recombinant target protein.

17. The method as claimed in claim 16 wherein the protein therapy is for the treatment of cystic fibrosis.

18. A pharmaceutical composition comprising a VLP as defined in claim 1 together with at least one pharmaceutically acceptable carrier, diluent or excipient.

19. (canceled)

20. (canceled)

21. A method for the production of a virus-like particle (VLP), said method comprising the coexpression of a viral structural protein, or fragment or derivative thereof, capable of forming an enveloped VLP, together with a fusiogenic protein, together a recombinant target protein in an in vitro cultured cell and isolating the VLP from the culture media.

22. An in vitro host cell comprising:
a) a nucleic acid encoding a viral structural protein, or fragment or derivative thereof, capable of forming an enveloped virus-like particle;
b) a nucleic acid encoding a fusiogenic protein; and
c) a nucleic acid encoding a recombinant target protein.

23. A nucleic acid vector comprising:
a) a sequence encoding a viral structural protein, or fragment or derivative thereof, capable of forming an enveloped virus-like particle;
b) a sequence encoding a fusiogenic protein; and
c) a cloning site in which the coding sequence of a target protein can be inserted.

24. The vector as claimed in claim 23 wherein the vector further comprises at least one from the following list: an origin of replication, a selectable marker, a transcriptional start site, a transcriptional enhancer, a transcriptional inducer, a transcriptional control element, a 3′ untranslated control sequence, a 5′ untranslated control sequence, sequences to allow for detection of the target protein product and sequences to allow for the purification of the target protein product.

25. A kit comprising a vector as defined in claim 23 and a host cell line.

26. A kit comprising:
(a) a stable host cell line comprising a nucleic acid encoding a viral structural protein, or fragment or derivative thereof, capable of forming an enveloped virus-like particle and a nucleic acid encoding a fusiogenic protein; and
(b) a vector suitable for transfection of said host cell line with a sequence encoding a target protein.

27. A virus-like particle (VLP) having a plasma membrane-derived lipid bilayer envelope, said VLP further comprising at least one viral structural protein, or fragment or derivative thereof, capable of forming an enveloped VLP and a recombinant membrane bound target protein, wherein the target protein is located in the envelope of the virus-like particle.

28. The VLP as claimed in claim 1 wherein the plasma membrane-derived lipid bilayer envelope comprises at least six different lipid types.

29. The VLP as claimed in claim 1 wherein the plasma membrane-derived lipid bilayer envelope comprises at least eight different lipid types.

30. The VLP as claimed in claim 1 wherein the VLP comprises at least 3000 copies of the viral structural protein.

31. The VLP as claimed in claim 1 wherein the VLP comprises at least 4000 copies of the viral structural protein.
